(19) United States
(12) Patent Application Publication
(10) Pub. No.: US 2013/0303622 A1
(43) Pub. Date: Nov. 14, 2013

(54) AQUEOUS ANTI-MICROBIAL SOLUTION CONTAINING CHLORHEXIDINE DIGLUCONATE AND TARTARIC ACID, METHOD FOR PREPARING SAME, AND ANTI-MICROBIAL ORTHODONTIC CEMENT

(71) Applicants: Marcel Marchiori Farret, Santa Maria (BR); Eduardo Martinelli Santayanna de Lima, Porto Alegre (BR); Hugo Mitsuo Silva Oshima, Porto Alegre (BR); Eduardo Goncalves Mota, Porto Alegre (BR)

(72) Inventors: Marcel Marchiori Farret, Santa Maria (BR); Eduardo Martinelli Santayanna de Lima, Porto Alegre (BR); Hugo Mitsuo Silva Oshima, Porto Alegre (BR); Eduardo Goncalves Mota, Porto Alegre (BR)

(73) Assignee: Uniao Brasileira de Educacao e Assistencia Mantenedora da PUC RS, Porto Alegre (BR)

(21) Appl. No.: 13/639,341

(22) PCT Filed: Apr. 5, 2011
(86) PCT No.: PCT/BR2011/000094

§ 371 Date: Jun. 3, 2013

(30) Foreign Application Priority Data
Apr. 5, 2010 (BR) PI1000977-9

(51) Int. Cl. A61K 6/00

(52) U.S. Cl. CPC A61K 6/0067
USPC 514/635

(57) ABSTRACT

The present invention deals with a method for the preparation of an antimicrobial aqueous solution containing chlorhexidine digluconate and tartaric acid for use with dental/orthodontic glass ionomer cement, and an antimicrobial aqueous solution substantially improved by the inclusion of chlorhexidine digluconate up to 18% and tartaric acid up to 11%.

### FIELD OF THE INVENTION

The present invention lies in the field of dentistry and orthodontics. Specifically, the present invention deals with a method for preparation of an aqueous solution for use as dental/orthodontic cement containing chlorhexidine digluconate and tartaric acid, and an aqueous solution for use as a dental/orthodontic cement containing chlorhexidine digluconate and tartaric acid.

### ANTECEDENTS OF THE INVENTION

At present, the materials most used for the cementing of orthodontic braces are conventional glass ionomer cements. These materials have qualities that make them the material of choice, such as biocompatibility, releasing of fluorine, and chemical bonding to the enamel of the teeth. However, the conventional glass ionomer cements present on the market have an antibacterial action against a very small spectrum of microorganisms and are not able to prevent the occurrence of pathologies near the orthodontic braces. These cements do not have a bactericidal effect, but only a bacteriostatic one. This bacteriostatic effect can last for a long period of time, but it acts only against a small quantity of bacteria existing in the oral cavity.

Patients undergoing orthodontic treatment normally require a cementing of braces and/or auxiliary devices with braces. These braces and the devices joined to them present sites for buildup of plaque in areas above and below the gum line, where dental hygiene becomes more complicated. Patients under orthodontic treatment commonly present caries and inflamed gums associated with the orthodontic braces and this can impede or prevent the progress of the treatment.

Thus, there is still a demand for a cement with greater antimicrobial activity for the cementing of orthodontic braces that is able to present a bacteriostatic and bactericidal effect, in order to maximize the results of the treatment performed.

In the specialized literature, some documents describe methods and products of dental use that utilize chlorhexidine digluconate. The references which surround the invention, yet without anticipating or even suggesting it, are listed below.

The article “Chlorhexidine-modified glass ionomer for band cementation? An in vitro study” (Journal of Orthodontics, Vol. 32, 2005, 36-42) refers to a process of inclusion of DGC in glass ionomer cements. The present invention differs from this document in its not mentioning the adding of tartaric acid. In the article, the authors performed tensile strength tests on orthodontic bands cemented on teeth, which is an interesting simulation of the buccal environment during orthodontic treatment, but it is not possible to infer solely by this analysis that the mechanical properties of this material were not affected by the inclusion of DGC and the present invention proves this fact. Therefore, it is not an obvious association, since other acids already exist that can be added to the material to initiate and accelerate the process of setting of the orthodontic cements. The use of tartaric acid has been shown to be important primarily for the preservation of the mechanical properties of the materials.

On the inclusion of chlorhexidine gluconate in glass ionomer cements referred to in the article in the Revista de Odontologia da UNESP. 2008; 37(1): 59-64: the materials mentioned in the article and by the references cited are all cements used for restorations or filling of cavities, and so are different from the cementing materials of the present invention. These materials have different formulations, both in powder and in liquid. Unlike what has been found by the authors, during the analysis of the present inventors no reduction was observed in the mechanical properties of the cements evaluated, proving the possibility of including chlorhexidine digluconate in the materials of the mentioned document.

In another article, Olympio, K. P. K et al., entitled “Prevention of dental caries and periodontal disease in orthodontics: an absolute necessity” (R Dental Press Ortodon Ortop Facial. Maringá, v. 11, n. 2, p. 110-119, March/April 2006), reference is made to varnishes that contain 10% chlorhexidine for use in areas of possible bacterial buildup during orthodontic treatments. The cited article differs from the present invention in not discussing materials for use in orthodontic gluing or cementing. In orthodontic braces, the principal occurrences of pathologies are the formation of caries in areas between the enamel surface and the brace and the development of periodontal disease in areas where the braces are below the gum line. In both situations, these varnishes could not be used. In these cases, the glass ionomer cement manipulated with aqueous solution of DGC of the present invention can be used for the cementing, solving these problems.

Document PI 9714943-8 refers to the inclusion of chlorhexidine digluconate in chewing gum to increase the control of plaque during the time of use of such gum. The present invention differs from this document by dealing with the inclusion of antimicrobial agents in glass ionomer cements to increase the control of bacterial plaque in areas close to the orthodontic braces and dental prostheses. The use of chewing gum is contraindicated in patients during orthodontic treatment, since it can promote damage to the parts of the apparatus.

Document PI 0502111-1 A2 refers to means of bacterial control that make use of various substances, including chlorhexidine. These means are proposed for use as mouthwash, dentifrice, or topical application. Hence, they differ in not dealing with materials for use in cementing and permanent presence in the buccal environment, near sites of plaque buildup, such as orthodontic braces.

Another example of the use of digluconate related to orthodontic apparatus are the documents ES 2137139(A1), ES2163995(A1) and WO 0122930(A1), which make use of a formulation containing chlorhexidine digluconate for the preparation of a medication, more specifically, a mouthwash or dental cream for the treatment of alterations that are frequent in patients with poor occlusion and alterations such as gingivitis who are using orthodontic apparatus, and it has nothing in common with the present invention.

In the article “Use of chlorhexidine in control of the number of streptococci of the mutans group and of lactobacilli in patients wearing a fixed orthodontic apparatus (in: XIII Meeting of Postgraduate Alumni in Orthodontics of the UFRJ, 2004, Florianópolis. Annals of the XIII Meeting of Postgraduate Alumni in Orthodontics of the UFRJ. Maringá: Dental Press Editora Ltda, 2004. v. 13) by YASSUDA-MATTOS, D. H.; SOUZA, M. M. G.; HIRATA JUNIOR, R., the authors analyzed the antibacterial action of chlorhexidine used as mouthwash/rinse in patients during orthodontic treatment, verifying the reduction of plaque formation. The present invention differs from this document in dealing with the inclusion of antimicrobial agents in glass ionomer cements, which is not ever suggested in the mentioned article at any time.

As emerges from the literature searched, no documents were found that anticipate or suggest the teachings of the present invention, so that the solution proposed here has novelty and inventive activity with regard to the prior art.

For the purpose of increasing the bacterial control against a broad spectrum of microorganisms, the present invention proposes a method for the preparation of an antimicrobial aqueous solution containing chlorhexidine digluconate (DCG) and tartaric acid (AT) for use as a glass ionomer cement which increases the antibacterial action for a long time against bacteria that cause dental caries and periodontal diseases. The chlorhexidine digluconate acts with bacteriostatic and bactericidal effect against practically all bacteria of the buccal cavity, that is, a broad spectrum of the microorganisms which compromise buccal health. The tartaric acid is fundamental to initiating and accelerating the setting process of the material, which is its essential use in the present invention for the preservation of the mechanical properties of the materials, since this acid is not used in related materials of the prior art with the purpose of preserving the mechanical properties of the materials.

### SUMMARY OF THE INVENTION

The present invention deals with a method for the preparation of an antimicrobial aqueous solution for use with dental/orthodontic glass ionomer cement containing chlorhexidine digluconate and tartaric acid for use as a glass ionomer cement, an antimicrobial aqueous solution containing chlorhexidine digluconate and tartaric acid for use with a glass ionomer cement, and an antimicrobial orthodontic cement containing chlorhexidine digluconate and tartaric acid.

One of the objects of the present invention is a method for preparation of an antimicrobial aqueous solution comprising the following steps:
a) preparing an aqueous liquid base with chlorhexidine digluconate up to around 18% by weight of the final liquid; and
b) adding to the liquid base of a) tartaric acid up to around 11% by weight of the final liquid.

The final weight of tartaric acid and DGC is around ⅓ of the quantity incorporated in the liquid.

The method of the present invention involves the addition of chlorhexidine digluconate up to around 18% by weight, preferentially 10 to 18% by weight, preferably 15 to 18% by weight, especially 18% by weight.

The method of the present invention preferentially comprises the adding of the tartaric acid up to around 11% by weight, preferentially 8 to 11% by weight, preferably 9 to 11% by weight, especially 10% by weight. Optionally, other acids could replace the tartaric acid, maintaining the same concentrations, such as itaconic acid or maleic acid.

Preferably, the present invention uses an aqueous solution that is to be used in combination with a solid base (preferentially a powder), which are to be mixed prior to use.

In a preferential embodiment, the chlorhexidine digluconate and the tartaric acid are added to the liquid base of the orthodontic cement.

One of the additional objects of the present invention is an antimicrobial aqueous solution comprising chlorhexidine digluconate and tartaric acid, produced by the method of the present invention.

The antimicrobial aqueous solution of the present invention comprises chlorhexidine digluconate up to around 18% by weight, preferentially 10 to 18% by weight, preferably 15 to 18% by weight, especially 18% by weight.

The solution of the present invention will preferentially comprise tartaric acid up to around 11% by weight, preferentially 8 to 11% by weight, preferably 9 to 11% by weight, especially 10% by weight. Optionally, other acids could replace the tartaric acid, maintaining the same concentrations, such as itaconic acid or maleic acid.

An additional object of the present invention is an antimicrobial orthodontic cement containing chlorhexidine digluconate and tartaric acid, prepared by the following steps of:

a) selecting an adequate dental/orthodontic glass ionomer cement;

b) adding the antimicrobial aqueous solution of the present invention.

The inclusion of the chlorhexidine digluconate in aqueous solution manipulated with glass ionomer cements for the cementing of orthodontic braces increased the bacterial control against a broad spectrum of microorganisms. The tartaric acid is responsible for initiating and accelerating the process of setting of the materials. Without the addition of the tartaric acid, the material has a longer setting process and loses some of its mechanical properties. Thus, the combination of the materials of the present invention in their respective proportions increased the bacterial control against a broad spectrum of microorganisms, while at the same time they do not alter the mechanical properties of these materials, suggesting that these materials can be clinically used with safety and adequate performance.

These and other objects of the invention will be immediately appreciated by those versed in the art and by companies with interests in this field, and shall be described in sufficient detail for their reproduction in the following specification.

### DETAILED SPECIFICATION OF THE INVENTION

In orthodontic treatment, patients frequently present with pathologies such as tooth decay and periodontal disease related to the accessories of the apparatus owing to the lack or difficulty of hygiene. The inclusion of DGC+AT in the cements can provide a better bacterial control in areas where the orthodontic braces will be positioned, and so it will be possible to reduce the incidence and severity of these pathologies.

The same method can be used for glass ionomer cements for restorations and filling of tooth cavities caused by decay. In addition, the method in question provides an increase in the microhardness of the material, which is clinically important for improving the mechanical properties of the material. Thus, when the present method is applied in restoration cements (dental and pediatric), there will be a considerable technical improvement, since it allows the material to have greater longevity with less wear due to occlusion and chewing.

Furthermore, in other types of cement, such as surgical cement, which are used in the dental field or other areas of health care and which require better bacterial control without loss of other properties, the proposal of including chlorhexidine digluconate can be extremely important in order to avoid infections.

In addition, any orthodontic cement can be used in the present invention, even other orthodontic cements not yet described are included in the scope of the present invention, as long as they present adequate characteristics so that the final product has the mechanical and microbiological properties set forth in this invention.

The preferential orthodontic cements used in the present invention, generally speaking, are prepared by a mixing of bases, where at least one of the bases is liquid and the other solid. In general, they include at least one solid base, usually a powder, and at least one liquid base, and so by the mixing of these bases one obtains the final product that will be used in the orthodontic cement. Cements having only liquid base(s) or cements with at least one gel base are included in the group of orthodontic cements that can be used in the present invention. Thus, for effects of this invention, the addition steps b) and c) of the present method should be understood as applying to gel bases, for cases of orthodontic cements with at least one gel base and without a liquid base.

In the present invention, the formula of glass ionomer cements for the cementing of orthodontic braces and prostheses has been substantially improved by the inclusion of chlorhexidine digluconate (DGC) up to 18% and tartaric acid (AT) up to 11% in the aqueous solution of the material.

The inclusion of antimicrobial agents in the aqueous solutions of the glass ionomer cements for temporary and permanent cementing has been realized, thus increasing the control of bacterial plaque in areas near the orthodontic braces and dental prostheses, that is, areas where plaque builds up.

Thus, a method is presented for the preparation of an antimicrobial aqueous solution for use with a dental/orthodontic cement, comprising the following steps:
a) preparing an aqueous liquid base with chlorhexidine digluconate up to around 18% by weight of the final liquid; and
b) adding to the liquid base of a) tartaric acid up to around 11% by weight of the final liquid.

Preferentially, the present invention uses an adequate orthodontic cement composed of at least one liquid base, or an adequate orthodontic cement composed of at least one solid base and at least one liquid base to be mixed prior to use; or an adequate orthodontic cement composed of at least one gel base.

The method of the present invention comprises the addition of the chlorhexidine digluconate up to around 18% by weight of the liquid, preferentially 10 to 18% by weight, preferably 15 to 18% by weight, especially 18% by weight.

The method of the present invention preferentially comprises the addition of the tartaric acid up to around 11% by weight, preferentially 8 to 11% by weight, preferably 9 to 11% by weight, especially 10% by weight. Optionally, other acids could replace the tartaric acid, maintaining the same concentrations, such as itaconic acid or maleic acid.

Preferably, the present invention uses an adequate orthodontic cement composed of a solid base and a liquid base to be mixed prior to use.

In one preferential embodiment, the chlorhexidine digluconate and the tartaric acid are added to the liquid base of the orthodontic cement.

In a nonlimiting manner, two conventional glass ionomer cements were selected (Ketac Cem—3M/ESPE and Voco—Meron) that are used for cementing of orthodontic braces and cementing of tooth crowns. These two cements were manipulated with their powder in the original composition and an added aqueous solution of DGC in two different concentrations (10 and 18%) with addition of tartaric acid (AT) 10% during the manipulation process. Thus, conventional glass ionomer cements were developed with chlorhexidine digluconate in the final formulation.

Mechanical tests were then set up to verify the mechanical properties (diametral tensile strength, compressive strength, surface microhardness and shear strength) of these cements in the original formulation (control) and with the addition of DGC (10 and 18%)+tartaric acid (AT) 10%, and bacterial culturing tests were set up to verify the antibacterial effect of these materials. All the tests performed are listed in tables 1 to 4.

In the mechanical tests, there was no statistically significant difference between the cements with and without the addition of DGC+AT.

Concentrations of DGC of up to 18% were tested, adding up to 10% of AT. It was not possible to use concentrations above 18% since the reagent is present in the liquid state with maximum concentration of 18%.

Concentrations below 5% DGC in the liquid represent 2.5% in the final formulation of the material (cement+liquid) and these concentrations were considered to be too low to obtain greater bacterial control.

Therefore, it is suggested to use concentrations above 5% and up to a maximum of 18% of DGC in the liquid of the material, adding AT up to 11%, preferentially 10%. This composition is a liquid. It is the liquid of the cement. After this, we mix with the powder of the cement and a solid material (cement) is formed by the setting process.

TABLE 1
Means, standard deviation, variance analysis and Tukey
test comparing the six cements to each other in terms
of compressive strength, measured in MPa.

| Material | n | Mean (Mpa) | SD |
|---|---|---|---|
| Ketac cem conventional | 12 | 52.04^{A} | 19.23 |
| Ketac cem 10% of DGC | 12 | 37.83^{ABC} | 12.71 |
| Ketac cem 18% of DGC | 12 | 47.73^{AB} | 18.71 |
| Voco Meron conventional | 12 | 38.09^{ABC} | 14.31 |
| Voco Meron 10% of DGC | 12 | 33.78^{BC} | 8.45 |
| Voco Meron 18% of DGC | 12 | 29.83^{C} | 8.13 |

* Means followed by the same letter do not differ from each other.

The results of Table 1 verify that there was no significant difference for the compressive strength values (p>0.05) between the control group (without chlorhexidine digluconate) and the groups including 10 and 18% of chlorhexidine digluconate in each of the two materials analyzed (Meron and Ketac Cem). There was only a significant difference between the two commercial brands (p<0.05). These results show that the inclusion of DGC did not alter the compressive strength of the materials, allowing DGC in the concentrations indicated to be used clinically with safety.

TABLE 2
Means, standard deviation, variance analysis and Tukey
test comparing the six cements to each other in terms
of diametral tensile strength, measured in MPa.

| Material | n | Mean (Mpa) | SD |
|---|---|---|---|
| Ketac cem conventional | 12 | 7.41 ^{A} | 1.72 |
| Ketac cem 10% of DGC | 12 | 6.93 ^{AB} | 1.89 |
| Ketac cem 18% of DGC | 12 | 7.24 ^{AB} | 1.90 |
| Voco Meron conventional | 12 | 6.05 ^{AB} | 1.60 |
| Voco Meron 10% of DGC | 12 | 5.75 ^{AB} | 1.22 |
| Voco Meron 18% of DGC | 12 | 5.36 ^{B} | 1.05 |

* Means followed by the same letter do not differ from each other.

The results of Table 2 verify that there was no significant difference for the diametral tensile strength values (p>0.05) between the control group (without chlorhexidine digluconate) and the groups including 10 and 18% of chlorhexidine digluconate in each of the two materials analyzed (Meron and Ketac Cem). There was only a significant difference between Ketac Cem conventional and Voco Meron with 18% DGC (p<0.05). The results showed that the inclusion of DGC did not alter the diametral tensile strength of the materials, allowing DGC to be used clinically in the concentrations indicated with safety of maintaining this property.

TABLE 3
Means, standard deviation, variance analysis and Tukey
test comparing the six cements to each other in terms
of surface microhardness, measured in MPa.

| Material | n | Mean (Mpa) | SD |
|---|---|---|---|
| Ketac cem conventional | 15 | 78.08^{B} | 14.03 |
| Ketac cem 10% of DGC | 15 | 97.62^{A} | 20.81 |
| Ketac cem 18% of DGC | 15 | 89.36^{AB} | 15.50 |
| Voco Meron conventional | 15 | 36.11^{C} | 6.35 |
| Voco Meron 10% of DGC | 15 | 43.84^{C} | 4.75 |
| Voco Meron 18% of DGC | 15 | 44.28^{C} | 6.82 |

* Means followed by the same letter do not differ from each other.

The results of Table 3 verify that there was a statistically significant increase of around 10 to 18% (p<0.05) in the microhardness of Ketac Cem with DGC added at 18 and 10% respectively, and an increase of around 10% in the microhardness of Voco Meron with the addition of the two concentrations of DGC, yet with no significant difference in regard to the control (p>0.05). The increase in the microhardness of both materials is very important from the standpoint of mechanical performance. Therefore, we can expect these materials to display better clinical performance when used for dental/orthodontic cementing.

TABLE 4
Means, standard deviation, variance analysis and Tukey test comparing
the six cements to each other in terms of shear strength*.

| Material | n | Mean (Mpa) | SD |
|---|---|---|---|
| Ketac cem conventional | 14 | 0.46 | 0.22 |
| Ketac cem 10% | 14 | 0.54 | 0.22 |
| Ketac cem 18% | 14 | 0.50 | 0.24 |
| Voco Meron conventional | 14 | 0.44 | 0.21 |
| Voco Meron 10% | 14 | 0.58 | 0.17 |
| Voco Meron 18% | 14 | 0.57 | 0.16 |

*There was no significant difference between the groups.

The results of Table 4 show that there was no statistically significant difference (p>0.05) between the conventional materials and the materials with addition of chlorhexidine digluconate for both the cements (Ketac Cem and Meron). The proof that the addition of DGC does not alter the shear strength shows that these materials can be used clinically. Therefore, one can infer that the materials will provide a bond similar for both enamel and the metal of the brace, maintaining a constant bond between these two, with no defects during the orthodontic treatment.

### Results of the Bacterial Culturing Tests

Tests of diffusion in agar were carried out for Streptococcus mutans, the principal cause of tooth decay. The cements were compared in their original compositions and the experimental groups with addition of 10 and 18% of chlorhexidine digluconate. The test bodies of the cements were prepared and immersed in distilled water that was changed daily, to simulate the buccal environment, and at the time of analysis they were placed in openings made in the culture mediums. The culture mediums (plates) were placed in an oven for 48 hours at 37° C. The bacterial growth inhibition haloes around the test bodies were then measured in periods of 5, 45 and 65 days from preparation of the test bodies, to verify if there was significant antibacterial action.

The bacterial culturing results are presented in tables 5, 6 and 7.

The results of the comparison between the control group (without DGC) and the two experimental groups (10 and 18% DGC) for the two cements (Ketac and Meron) showed that the experimental groups presented a greater inhibition of bacterial growth (p<0.05) with a statistical difference, since the groups did not present any inhibition of bacterial growth. The experimental group with addition of 18% DGC presented 30 to 50% more inhibition in the first analysis (5 days) as compared to the group with 10% addition. Already in the analysis at 65 days there was 50 to 100% more inhibition for the group with 18% (Table 5).

In the comparison between the two materials (Ketac and Meron), it is verified that there was little difference between them (table 6).

In the comparison between the three analysis periods (5, 45 and 65 days) it was verified that there was a definite decrease in the antibacterial activity, principally for the cement Meron, which showed a 30% reduction for both 10 and 18% addition. The cement Ketac did not present a significant reduction for the group with addition of 18% DGC (10% reduction), and the group with 10% DGC presented a reduction of 35% in the antibacterial activity.

TABLE 5
Comparison among the subgroups Normal, 10% and 18% for each
material in each period of time according to the measurement of
the inhibition haloes in mm (n = 6 cases for each subgroup)

| | | Material Ketac | | | Material Meron | | |
|---|---|---|---|---|---|---|---|
| Period | Subgroup | Mean | SD | p | Mean | SP | p |
| 5 days | Normal | 10.55^{A} | 1.55 | 0.000 | 0.00^{A} | 0.00 | 0.000 |
| | 10% | 15.64^{B} | 1.42 | | 12.22^{B} | 0.91 | |
| | 18% | 20.63^{C} | 1.84 | | 20.12^{C} | 0.81 | |
| 45 days | Normal | 0.00^{A} | 0.00 | 0.000 | 0.00^{A} | 0.00 | 0.000 |
| | 10% | 10.69^{B} | 0.43 | | 9.61^{B} | 0.86 | |
| | 18% | 18.31^{C} | 2.81 | | 16.01^{C} | 1.45 | |
| 65 days | Normal | 0.00^{A} | 0.00 | 0.000 | 0.00^{A} | 0.00 | 0.000 |
| | 10% | 9.39^{B} | 0.40 | | 9.58^{B} | 0.14 | |
| | 18% | 18.21^{C} | 0.72 | | 13.16^{C} | 0.44 | |

*Means followed by the same letter do not differ from each other
**SD = standard deviation

TABLE 6
Comparison between the materials Ketac and Meron for each subgroup
in each period of time according to the measurement of the inhibition
haloes in mm (n = 6 cases for each material)

| Subgroup | Period | Material | Mean | SD | P |
|---|---|---|---|---|---|
| Normal | 5 days | Ketac | 10.55 | 1.55 | 0.002 |
| | | Meron | 0.00 | 0.00 | |
| | 45 days | Ketac | 0.00 | 0.00 | ** |
| | | Meron | 0.00 | 0.00 | |
| | 65 days | Ketac | 0.00 | 0.00 | ** |
| | | Meron | 0.00 | 0.00 | |
| 10% | 5 days | Ketac | 15.64 | 1.42 | 0.002 |
| | | Meron | 12.22 | 0.91 | |
| | 45 days | Ketac | 10.69 | 0.43 | 0.041 |
| | | Meron | 9.61 | 0.86 | |
| | 65 days | Ketac | 9.39 | 0.40 | 0.240 |
| | | Meron | 9.58 | 0.14 | |
| 18% | 5 days | Ketac | 20.63 | 1.84 | 0.818 |
| | | Meron | 20.12 | 0.81 | |
| | 45 days | Ketac | 18.31 | 2.81 | 0.132 |
| | | Meron | 16.01 | 1.45 | |
| | 65 days | Ketac | 18.21 | 0.72 | 0.002 |
| | | Meron | 13.16 | 0.44 | |

** It was not possible to perform a statistical test

TABLE 7
Comparison among the periods of 5, 45 and 65 days for each
subgroup and for each material according to the measurement
of the inhibition haloes in mm (n = 6 cases for each period)

| | | Material Ketac | | | Material Meron | | |
|---|---|---|---|---|---|---|---|
| Subgroup | Period | Mean | SD | p | Mean | SD | p |
| Normal | 5 days | 10.55^{A} | 1.55 | 0.002 | 0.00 | 0.00 | ** |
| | 45 days | 0.00^{B} | 0.00 | | 0.00 | 0.00 | |
| | 65 days | 0.00^{B} | 0.00 | | 0.00 | 0.00 | |
| 10% | 5 days | 15.64^{A} | 1.42 | 0.002 | 12.22^{A} | 0.91 | 0.011 |
| | 45 days | 10.69^{B} | 0.43 | | 9.61^{B} | 0.86 | |
| | 65 days | 9.39^{C} | 0.40 | | 9.58^{B} | 0.14 | |
| 18% | 5 days | 20.63 | 1.84 | 0.135 | 20.12^{A} | 0.81 | 0.002 |
| | 45 days | 18.31 | 2.81 | | 16.01^{B} | 1.45 | |
| | 65 days | 18.21 | 0.72 | | 13.16^{C} | 0.44 | |

*Means followed by the same letter do not differ from each other
** SD = standard deviation

Based on the results obtained, it is possible to affirm that the addition of DGC in glass ionomer cements for cementing of orthodontic braces promotes a significant inhibition of bacterial growth, including in the long term. Therefore, this combination is an alternative for use in cementing of orthodontic braces for a better control of the formation of plaque, development of caries and periodontal disease.

The present invention also contemplates an aqueous solution to be used with dental/orthodontic glass ionomer cement comprising chlorhexidine digluconate and tartaric acid, prepared by the method of the present invention. The present invention can be used in any type of dental/orthodontic cement, such as surgical, bone restoration, and other cements known in the prior art.

The antimicrobial aqueous solution of the present invention contains the chlorhexidine digluconate up to around 18% by weight, preferentially 10 to 18% by weight, preferably 15 to 18% by weight, especially 18% by weight in the final formulation (cement+liquid).

The antimicrobial aqueous solution of the present invention contains the tartaric acid up to around 11% by weight, preferentially 8 to 11% by weight, preferably 9 to 11% by weight, especially 10% by weight in the final formulation (cement+liquid).

Depending on the application, the aqueous solution can entirely replace the solvents used with cement, preferentially the powder cements, or it can be combined with the solvent of the cement. This will result in different concentrations of DGC and AT in the final product, providing various degrees of control of microorganisms.

The mentioned solution can be made available, without being limited in this way, in vials, capsules, sachets or other means. It can also be made available in the form of a kit made up of two vials (cement+liquid), two capsules, two sachets, other means and/or combinations of these.

The presentation of the product can be done, without being limited to this, in definite doses with various concentrations, for example, two capsules, one containing a particular quantity of the solution and another containing a particular quantity of the cement, which when mixed will produce a final antimicrobial cement with a particular concentration of DGC and AT. In this way, various types of packages could be made available so that when mixed one obtains a final antimicrobial cement with the desired concentration. Another product presentation involves large packages, where the reagents will be used as desired for a particular concentration.

Thus, the present invention provides for the preparation of an antimicrobial orthodontic cement comprising chlorhexidine digluconate and tartaric acid, produced by the use of the antimicrobial aqueous solution of the present invention in combination with an orthodontic cement.

The present antimicrobial orthodontic cement containing chlorhexidine digluconate and tartaric acid is prepared by the steps of:
a) selecting an adequate dental/orthodontic glass ionomer cement;
b) adding the antimicrobial aqueous solution of the present invention.

The antimicrobial orthodontic cement of the present invention contains the chlorhexidine digluconate up to around 10% by weight, preferentially 5 to 10% by weight, preferably 7.5 to 10% by weight, especially 9% by weight in the final formulation (cement+liquid).

The antimicrobial orthodontic cement of the present invention contains the tartaric acid up to around 5.5% by weight, preferentially 4 to 5.5% by weight, preferably 4.5 to 5.5% by weight, especially 5% by weight in the final formulation (cement+liquid).

The final concentration of the chlorhexidine digluconate and the tartaric acid in the final antimicrobial orthodontic cement is decreased, since upon mixing the cement powder with the present aqueous solution there occurs a dilution of the reagents, obtaining a final antimicrobial orthodontic cement suitable for the use.

The examples shown here have the purpose of merely exemplifying one of the countless ways of implementing the invention, yet without limiting its scope. Those versed in the art will appreciate the teachings presented here and will be able to reproduce the invention in the embodiments presented and in other variants, coming under the scope of the appended claims.

1. A method for preparation of an antimicrobial aqueous formulation, comprising the following steps:
a) preparing an aqueous liquid base with chlorhexidine digluconate in an amount up to about 18% by weight of the final formulation; and
b) adding to the liquid base of step a) tartaric acid in an amount up to about 11% by weight of the final formulation.

2. The method according to claim 1, wherein the final weight of tartaric acid and chlorhexidine digluconate is about one-third of the quantity incorporated in the final formulation.

3. The method according to claim 1, comprising the addition of chlorhexidine digluconate in an amount up to 18% by weight of the final formulation.

4. The method according to claim 3, comprising the addition of chlorhexidine digluconate in an amount of 15 to 18% by weight of the final formulation.

5. The method according to claim 4, comprising the addition of chlorhexidine digluconate in an amount of 18% by weight of the final formulation.

6. The method according to claim 1, comprising the addition of tartaric acid in an amount up to 11% by weight of the final formulation.

7. The method according to claim 6, comprising the addition of tartaric acid in an amount of 8 to 11% by weight of the final formulation.

8. The method according to claim 7, comprising the addition of tartaric acid in an amount of 9 to 11% by weight of the final formulation.

9. The method according to claim 8, comprising the addition of tartaric acid in an amount of 10% by weight of the final formulation.

10. The method according to claim 1, comprising adding the chlorhexidine digluconate to the liquid base for use as a gel of an orthodontic cement.

11. The method according to claim 1, comprising adding the tartaric acid to the liquid base for use as a gel of an orthodontic cement.

12. An antimicrobial aqueous formulation, comprising chlorhexidine digluconate and tartaric acid.

13. The formulation according to claim 12, wherein the chlorhexidine digluconate is present in an amount up to 18% by weight of the final formulation.

14. The formulation according to claim 13, wherein the chlorhexidine digluconate is present in an amount of 15% to 18% by weight of the final formulation.

15. The formulation according to claim 14, wherein the tartaric acid is present in an amount of up to 11% by weight of the final formulation.

16. The formulation according to claim 15, wherein the tartaric acid is present in an amount of 5% to 11% by weight of the final formulation.

17. The formulation according to claim 16, wherein the tartaric acid is present in an amount of 10% by weight of the final formulation.

18. An antimicrobial orthodontic cement formulation, comprising:
a) at least one dental/orthodontic glass ionomer cement; and
b) an antimicrobial aqueous solution containing chlorhexidine digluconate and tartaric acid.

19. The cement formulation according to claim 18, the cement is formulation selected from the group consisting of: cements composed of at least one liquid base; cements composed of at least one solid base and at least one liquid base to be mixed prior to use; and cements composed of at least one gel base.

20. The cement formulation according to claim 18, comprising chlorhexidine digluconate in an amount up to 10% by weight of the final cement formulation.

21. The cement formulation according to claim 20, comprising chlorhexidine digluconate in an amount of 7.5% to 10% by weight the final cement formulation.

22. The cement formulation according to claim 21, comprising chlorhexidine digluconate in an amount of 9% by weight of the final cement formulation.

23. The cement formulation according to claim 18, comprising tartaric acid in an amount up to 5.5% by weight of the final cement formulation.

24. The cement formulation according to claim 23, comprising tartaric acid in an amount of 4% to 5.5% by weight of the final cement formulation.

25. The cement formulation according to claim 24, comprising tartaric acid in an amount of 5% by weight of the final cement formulation.
